(19) United States
(12) Patent Application Publication
(10) Pub. No.: US 2005/0118265 A1
(43) Pub. Date: Jun. 2, 2005

(54) Antifungal oral dosage forms and the methods for preparation

(76) Inventors: Anandi Krishnan, Mumbai (IN); Nilendu Sen, Mumbai (IN); Shirkant Bhonsle, Mumbai (IN); Kavita Chandurkar, St.John's (CA)

Correspondence Address: KENYON & KENYON, ONE BROADWAY, NEW YORK, NY, 10004, US

(21) Appl. No.: 10/781,997

(22) Filed: Feb. 19, 2004

(30) Foreign Application Priority Data
Nov. 28, 2003 (IN) 1231/MUM/2003

(51) Int. Cl. A61K 31/724; A61K 31/496; A61K 9/26

(52) U.S. Cl. 424/469; 514/58; 514/254.07; 264/109

(57) ABSTRACT

The present invention relates to pharmaceutical dosage forms which include an antifungal having poor solubility. The pharmaceutical dosage forms of the present invention further comprise non-spherical granules, which do not contain a coated core region and may be formed into pharmaceutically acceptable dosage forms. The antifungal active pharmaceutical ingredients include itraconazole, saperconazole, ketoconazole, voriconazole and fluconazole.

### CLAIM FOR PRIORITY

This application claims priority from Indian provisional application number 1231/MUM/2003 filed Nov. 28, 2003. The priority application is incorporated by reference herein in its entirety.

### FIELD OF THE INVENTION

The present invention relates to pharmaceutical dosage forms which include an antifungal having poor solubility. The pharmaceutical dosage forms of the present invention further comprise non-spherical granules which do not contain a coated core region which may be formed into pharmaceutically acceptable dosage forms.

### BACKGROUND OF THE INVENTION

The development of efficacious pharmaceutical compositions of azole antifungals, (e.g. itraconazole) is hampered considerably by the fact that these antifungals are only sparingly soluble in water. Itraconazole is a synthetic triazole antifungal agent that is used in the treatment of fungal infections, such as aspergillosis, blastomycosis, histoplasmosis, and fungal infection localized to the toenails and fingernails (onychomycosis). Itraconazole is a 1:1:1:1 racemic mixture of four diastereomers (two enantiomeric pairs), each possessing three chiral centers. It is represented by the following nomenclature: (±)-1-[(R*)-sec-butyl]-4-[p-[4-[p-[[(2R*,4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Ä2-1,2,4-triazolin-5-one mixture with (±)-1-[(R*)-sec-butyl]-4-[p-[4-[p-[[(2S*,4R*)-2-(2,4-dichlorophenyl)-2-(1H-1,2,4-triazol-1-ylmethyl)-1,3-dioxolan-4-yl]methoxy]phenyl]-1-piperazinyl]phenyl]-Ä 2-1,2,4-triazolin-5-one or (±)-1-[(RS)-sec-butyl]-4-[p-[4-[p-[[(2R,4S)-2-(2,4-dichlorophenyl)-2-(1H-1,2,4-triazol-1-ylmethyl)-1,3-dioxolan-4-yl]methoxy]phenyl]-1-piperazinyl]phenyl]-Ä 2-1,2,4-triazolin-5-one.

Itraconazole has a molecular formula of C₃₅H₃₈Cl₂N₈O₄ and a molecular weight of 705.64. It is a white to slightly yellowish powder that is insoluble in water, very slightly soluble in alcohol, and freely soluble in dichloromethane. It has a pKa of 3.70 (based on extrapolation of values obtained from methanolic solutions) and a log (n-octanol/water) partition coefficient of 5.66 at pH 8.1.

Itraconazole can be dissolved in methylene chloride and spray dried, fluid bed granulated or centrifugally granulated under controlled drying conditions. This yields an amorphous form of itraconazole. Itraconazole can also be dissolved with a water soluble, pH-independent polymer using non-aqueous solvents like methylene chloride, chloroform, ethanol or methanol. These solvents can be problematic, for example, the organic volatile impurities [OVI] limits for methylene chloride are extremely stringent. Extensive heating and drying steps are essential to bring down the limits of methylene chloride to recommended levels. Methylene chloride is known to be a health hazard. The liver and skin are also susceptible to acute effects from methylene chloride exposure. Chlorinated hydrocarbons as a class, which includes methylene chloride, are generally toxic to the liver.

U.S. Pat. No. 5,633,015 to Gillis, et al., discloses a pharmaceutical formulation for itraconazole and saperconazole in the form of beads. The beads comprise a central, rounded or spherical core, a coating film, and a seal-coating polymer layer. The core has a diameter of about 600 to about 700 μm (25-30 mesh). The coating film contains a hydrophilic polymer (such as hydroxypropyl methylcellulose) and a drug (e.g., itraconazole and/or saperconazole). The seal-coating polymer layer is applied to the drug coated cores to prevent sticking of the beads, which would have the undesirable effect of a concomitant decrease of the dissolution rate and of bioavailability. The beads use polyethylene glycol (PEG), in particular, PEG 20,000, as the seal-coating polymer.

U.S. Pat. No. 5,707,975 to Francois, et al., relates to formulations for oral administration which comprise an antifungal active ingredient, a sufficient amount of a cyclodextrin or derivative thereof as a solubilizer, an aqueous acidic medium as bulk liquid carrier and an alcoholic co-solvent. The pharmaceutical dosage form comprises a minimal volume of air above the solution, preferably, an inert gas. Liquid dosage forms are considered to be less stable, in general, when compared to solid dosage forms for oral administration and may also have shorter shelf lives.

U.S. Pat. No. 6,039,981 to Woo, et al., relates to a fused mixture of itraconazole and phosphoric acid. The process involves heating the mixture to a temperature of 100 to 170° C. However, the manufacturing process of the solid dispersion is hampered by a number of difficulties in controlling various process variables.

U.S. Pat. No. 6,663,897 to Namburi, et al., relates to a process for the formulation of itraconazole dosage forms. The itraconazole and a water soluble film forming polymer are dissolved in an acidified ethanolic solution and this solution is sprayed onto beads to obtain cores having a coating film comprising a water soluble polymer and itraconazole. In this invention the molar concentration of acid used is in the range of 1 to 3 moles and the coating solution is an 8% w/w solution.

International Publication No. WO 01/85135, relates to a process in which itraconazole and a water soluble pH independent polymer are dissolved in a combination of solvents like methylene chloride, chloroform, ethanol or methanol. They are then spray dried to obtain fine particles which are then compressed into suitable dosage forms. The disadvantage of this process is that large quantities of potentially hazardous solvents are involved and may be a health hazard.

Accordingly, there remains a need for an improved process in making orally administrable pharmaceutical dosage forms including an antifungal that has poor solubility. Additionally, there remains a need for improved dosage forms including non-spherical granules which do not contain a coated core region.

### SUMMARY OF THE INVENTION

The present invention provides pharmaceutical dosage forms including an antifungal active pharmaceutical ingredient that has low solubility in aqueous media. Moreover, the present invention provides pharmaceutical dosage forms including non-spherical granules which do not contain a coated core region.

The present invention also provides for a pharmaceutical composition comprising a plurality of non-spherical granules, wherein the granules do not contain a coated core region. In the most preferred embodiment the antifungal active pharmaceutical ingredient is distributed uniformly throughout the non-spherical granule.

The granules of the invention comprise: an antifungal active pharmaceutical ingredient; a bulking agent; a disintegrant; a binding agent; and an acid. Preferably the antifungal active pharmaceutical ingredient is selected from the group consisting of itraconazole, saperconazole, ketoconazole, voriconazole and fluconazole.

One aspect of the present invention provides a method for preparing a pharmaceutical dosage form for administration to a patient in need thereof by dissolving an antifungal active pharmaceutical ingredient in alcohol, acid, and purified water. A bulking agent is mixed with a disintegrant and a binding agent. This mixture is granulated by the solution of the dissolved active agent to form non-spherical granules. The granules of the present invention are non-spherical and do not contain a coated core region. In addition, one advantage of the granules of the present invention is that they do not require a seal coating layer. The non-spherical granules are then formed into a pharmaceutical dosage form, for example, a tablet or capsule.

The active pharmaceutical ingredient (API) of the present invention is an antifungal agent. Preferably, the antifungal API of the present invention includes, but is not limited to, itraconazole, saperconazole, ketoconazole, voriconazole and fluconazole.

In a preferred embodiment of the present invention, the antifungal active pharmaceutical ingredient is itraconazole. Itraconazole is known to have poor solubility in aqueous media. However, the present invention dissolves the itraconazole in ethanol, concentrated hydrochloric acid, and purified water. It is then added to a mixture of mannitol, croscarmellose sodium, and polyvinyl pyrrolidone K25, or in an alternative, microcrystalline cellulose, croscarmellose sodium, and polyvinyl pyrrolidone K25. The mixture is then granulated, which can then be formed into various pharmaceutical dosage forms.

Another aspect of the present invention is a pharmaceutically acceptable dosage form comprising non-spherical granules with an antifungal active pharmaceutical ingredient, an acid, a bulking agent, a disintegrant, and a binding agent.

Another embodiment of the present invention provides methods of making pharmaceutical dosage forms including an active pharmaceutical ingredient.

Another aspect of the present invention is a pharmaceutically acceptable dosage form comprising non-spherical granules including an active antifungal pharmaceutical ingredient, an acid, a cyclodextrin, a bulking agent, a first disintegrant, a second disintegrant, a third disintegrant, and a binding agent. The three disintegrants may or may not be the same ingredient.

In another aspect of the present invention, a method for treatment of fungal infections, such as aspergillosis, blastomycosis, histoplasmosis, and fungal infection localized to the toenails and fingernails (onychomycosis), is provided. The method includes administering an effective amount of a composition of the present invention to a patient in need thereof.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

As used herein the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONI™ f127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

As used herein, the term “glidant” is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

As used herein, the term “lubricant” is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients (3^{rd} Ed. 2000), which are incorporated by reference herein.

### DETAILED DESCRIPTION OF THE INVENTION

The present invention relates to methods of manufacturing orally administrable pharmaceutical dosage forms which include an antifungal active pharmaceutical ingredient. The present invention includes improved methods for dissolving antifungals, some of which are known to be difficult to dissolve. This also allows for improved dosage forms that include the antifungal agent.

The present invention also provides for a pharmaceutical composition comprising a plurality of non-spherical granules, wherein the granules do not contain a coated core region. In the most preferred embodiment the antifungal active pharmaceutical ingredient is distributed uniformly throughout the non-spherical granule.

The granules of the invention comprise: an antifungal active pharmaceutical ingredient; a bulking agent; a disintegrant; a binding agent; and an acid. Preferably the antifungal active pharmaceutical ingredient is selected from the group consisting of itraconazole, saperconazole, ketoconazole, voriconazole and fluconazole.

In one embodiment of the present invention, a pharmaceutical dosage form is provided including non-spherical granules with an antifungal active pharmaceutical ingredient, an acid, a bulking agent, a disintegrant, and a binding agent. The preferred dosage forms of the present invention include, but are not limited to, tablets, capsules, and caplets.

The active pharmaceutical ingredient of the present invention is an antifungal agent. Preferably, the antifungal API of the present invention includes, but is not limited to, itraconazole, saperconazole, ketoconazole, and fluconazole. The preferred antifungal active pharmaceutical ingredient of the present invention is itraconazole. It is preferred to use a form of micronized API that has a particle size of less than about 50 microns with a particle size distribution of about 90% below 50 microns. This allows the micronized API to dissolve faster. It also allows for greater uniformity in the API lots. The granules formed by the present invention are non-uniform and non-spherical or they have an irregular or undefined shape or structure.

A preferred concentrated acid of the present invention is concentrated hydrochloric acid [HCl]. The molar ratio of antifungal agent to concentrated hydrochloric acid may be about 1:3.5. More preferably, the Hydrochloric acid of the present invention is HCl U.S.P. containing 36-38% w/w of HCl. Hydrochloric acid has a molecular weight of 36.5. Concentrated hydrochloric acid occurs as a clear colorless, fuming aqueous solution of hydrogen chloride with a pungent odor. Its functional category is as an acidifying agent, see, for example, Handbook of Pharmaceutical Excipients, Third Edition by Raymond C. Rowe et al., which is incorporated by reference herein. The antifungal agent is not soluble in hydrochloric acid alone.

A preferred alcohol of the present invention is ethanol. The ethanol is combined with the concentrated hydrochloric acid and purified water to form an ethanolic acid medium, which is used to dissolve the antifungal API. Ethanol is a common solvent in pharmaceutical formulations, but itraconazole is poorly soluble in ethanol. At the concentrations used in the present invention, this quantity alone is insufficient to dissolve the drug. The use of acidified ethanol is essential for the antifungal agent to be soluble.

The present invention uses ethanolic acid to dissolve the antifungal agent, but does not use a water soluble polymer as an essential component. Further, the present invention obtains the dissolution of the antifungal API using a water soluble monomer, preferably mannitol, in the weight ratio of from about 50 to about 70 percent weight of the final composition. The antifungal API may be dissolved in the alcohol, concentrated acid, and purified water.

A mixture of a bulking agent, a disintegrant, and a binding agent are prepared separately from the dissolved antifungal active agent solution. A preferred bulking agent of the present invention is mannitol. More preferred is D-mannitol. It is a hexahydric alcohol related to mannose and is isomeric with sorbitol. It is a water soluble monomer having no film forming properties. It is used as a sweetening agent and a diluent in tablets and capsules. In the present invention it is used as a bulking agent, and also has properties of a solubility enhancer for the antifungal API.

Another preferred bulking agent is microcrystalline cellulose. Microcrystalline cellulose is purified, partially depolymerized cellulose. It is a hydrophilic water insoluble polymer and has no film forming properties. In the present invention, its role is as a bulking agent for the active ingredient.

A preferred disintegrant of the present invention is croscarmellose sodium. Croscarmellose sodium is a cross-linked polymer of carboxymethyl cellulose sodium. It is a hydrophilic water insoluble polymer, which is used in pharmaceutical preparations as a disintegrant. The croscarmellose sodium in the present invention does not have film forming properties.

A preferred binding agent of the present invention is polyvinyl pyrrolidone (PVP), and a more preferred binding agent is PVP K25. Polyvinyl pyrrolidone is a synthetic polymer comprised essentially of linear 1-vinyl-2-pyrrolidone groups. The degree of polymerization results in polymers of various molecular weights. The PVP K25 has a molecular weight of about 30,000 units. In the present invention, the weight by weight ratio of antifungal agent to PVP K25 is about 1:0.12 or about 10:1.2. At this level, the role played is not that of a film forming agent or solubility enhancer, but rather, as a binding agent in the formation of granules. In the present invention, it is preferred that the weight by weight percentage of itraconazole is about 21.74% and that of the hydrophilic water soluble polymer is about 2.6%. At these concentrations, no film forming properties are attributed to the polymer. Rather, its role is as a binding agent in the formation of granules.

In another embodiment of the present invention, a pharmaceutically acceptable dosage form is provided including an antifungal active pharmaceutical ingredient, an acid, a cyclodextrin, a bulking agent, a first disintegrant, a second disintegrant, a third disintegrant, and a binding agent. The first, second, and third disintegrants may or may not be the same ingredient.

According to the present invention, preferred cyclodextrins may be selected from the group consisting of γ-cyclodextrin, β-cyclodextrin and derivatives thereof. More preferably the cyclodextrin of the present invention is hydroxypropyl-β-cyclodextrin (HP3-β-CD). HP-β-CD belongs to the class of cyclodextrins which are cyclic oligosaccharides containing at least 6 D-(+)-glucopyranose units attached α (1→4) glucoside bonds. The β-cyclodextrin contains 7 glucose units. As per their ordinary definition, polymers are large molecules consisting of repeated chemical units (‘mers’) joined together, usually in a line, like beads on a string. Each ‘mer’ is typically made up of more than 5 and less than 500 atoms. The word ‘polymer’ is applied when there are more than about 500 ‘mers’ stuck together. Polymeric molecules do not have well defined molecular weights. HP3-β-CD used herein has a molecular weight of 1309. The HP3-β-CD in the present invention is not a polymer and does not have film forming properties.

A preferred disintegrant of the present invention is crospovidone. Crospovidone is a white, free flowing, compressible powder that is a synthetic homopolymer of cross-linked N-vinyl-2-pyrrolidone.

A preferred lubricant of the present invention is magnesium stearate. Magnesium stearate is a common lubricant. It works in concentrations of about 0.5% to about 5%, although it is best to use it in the lowest effective concentration. Overblending the magnesium stearate can cause compaction problems.

The methods and pharmaceutical dosage forms of the present invention may further include pharmaceutically acceptable excipients, binders, glidants, lubricants, and/or diluents, fillers, such as lactose, starches, glucose, sucrose, mannitol, and silicic acid, lubricants such as talc, calcium stearate, magnesium stearate, solid polyethylene glycols, sodium lauryl sulfate, and mixtures thereof. The processes and pharmaceutical dosage forms of the present invention may also contain other required pharmaceutically acceptable excipients. The pharmaceutically acceptable excipients used in the present invention include fillers, glidants and lubricants that are typically used in the pharmaceutical arts for oral solid dosage forms. The filler used herein is inert filler, and may be water soluble or water insoluble fillers selected from those typically used in the pharmaceutical art for oral solid dosage forms. Examples include calcium carbonate, dicalcium phosphate, tricalcium phosphate, microcrystalline cellulose, monosaccharide, disaccharides, polyhydric alcohols, sucrose, dextrose, lactose, fructose, mannitol, sorbitol, alone or mixtures thereof and the like or mixtures thereof.

In one embodiment of the present invention, the antifungal active pharmaceutical ingredient is dissolved in a concentrated acid, an alcohol, and purified water. A mixture of a bulking agent, a disintegrant, and a binding agent are also combined. This mixture is then granulated by solution of the dissolved active antifungal. The resulting granules can be used to form acceptable pharmaceutical dosage forms, for example, filled into capsules or pressed into tablets. By following this method, the purified water and the ethanol will not appear in the final product.

The antifungal active pharmaceutical ingredient is dissolved in a mixture of a concentrated acid, an alcohol, and purified water to obtain a 15-35% w/w solution of the active pharmaceutical ingredient. This mix of solvents helps alleviate the poor solubility of the active ingredient. For example, itraconazole has difficulty dissolving in any one of the solvents alone, however, when the solvents are combined, itraconazole dissolves more readily.

In a preferred embodiment of the present invention, the itraconazole is dissolved in a mixture of ethanol, concentrated hydrochloric acid (37%) and purified water. Mannitol-D is added to croscarmellose sodium and PVP K25, and mixed well. This mixture is then granulated by the dissolved active agent by top spray technique using a fluid bed granulator. The resulting granules can then be directly filled into capsules or compressed into tablets.

In another preferred embodiment, itraconazole is dissolved in a mixture of ethanol, concentrated hydrochloric acid (37%) and purified water. Microcrystalline cellulose is added to croscarmellose sodium and PVP K25, and mixed well. This mixture is then granulated by the dissolved active agent by top spray technique using a fluid bed granulator. The resulting granules can then be directly filled into capsules or compressed into tablets.

In another embodiment of the present invention, a cyclodextrin is added to the dissolved active agent solution. More specifically, the antifingal active pharmaceutical ingredient is dissolved in a mixture of concentrated acid, alcohol, and purified water to form a dissolved active agent solution. A cyclodextrin is dissolved in purified water. The cyclodextrin solution is added to the dissolved active agent solution and they are mixed together. A bulking agent, a disintegrant, and a binding agent are mixed together. This mixture is then added to the dissolved active agent solution to form non-spherical granules. A disintegrant and a lubricant may be added to the granules.

In a preferred embodiment of the present invention, itraconazole is dissolved in a mixture of ethanol, concentrated hydrochloric acid (37%), and purified water. HP3-β-CD is dissolved in purified water. The HP3-β-CD solution is then added to the dissolved active agent solution and mixed well. Microcrystalline cellulose, croscarmellose sodium, crospovidone or a 1:1 ratio mixture of croscarmellose sodium and crospovidone and PVP K25 are mixed together. This mixture is then granulated by the dissolved active agent and cyclodextrin solution by top spray technique using a fluid bed granulator. Crospovidone, a second disintegrant, and magnesium stearate are added together to the non-spherical granules and then roll compacted. The roll compacted mass is then sized and milled. Crospovidone, a third disintegrant, is then added to those granules, and the granules are filled into capsules or compacted into tablets.

In another embodiment of the present invention, a method for treatment of fungal infections, for example, aspergillosis, blastomycosis, histoplasmosis, and fungal infection localized to the toenails and fingernails (onychomycosis) is contemplated by administering an effective amount of a composition of the present invention to a patient in need thereof.

The following examples are provided to enable one skilled in the art to practice the invention and are merely illustrative of the invention. The examples should not be read as limiting the scope of the claims.

### EXAMPLE 1

Table 1 Lists the Formula Used in Example 1

TABLE 1
Quantitative Formula

| | | Quantity per | |
|---|---|---|---|
| S.No: | Ingredients | dose [mg] | % w/w |
| 1. | Itraconazole | 100 | 21.74 |
| 2. | Mannitol | 302 | 65.65 |
| 3. | Croscarmellose sodium | 46.0 | 10.0 |
| 4. | Polyvinyl pyrrolidone K25 | 12.0 | 2.60 |
| 5. | Conc. Hydrochloric acid [37%] | 0.0415 ml | Molar ratio to |
| | | [0.04897 g | drug 1:3.5 moles |
| | | 48.97 mg] | |
| 6. | Ethanol* | — | — |
| 7. | Purified Water* | — | — |
| | Total | 460.0 | |

*Does not appear in final product.

Brief Process of the Invention:
1. Itraconazole is dissolved in the mixture of ethanol, concentrated hydrochloric acid (37%) and purified water.
2. Mannitol, croscarmellose sodium and polyvinyl pyrrolidone K25 are added together and mixed well.
3. The ingredients of step 2 are mixed well and then granulated by the solution of step 1 by top spray technique using a fluid bed granulator.
4. The granules thus obtained can be directly filled into capsules or can be compressed into tablets.

### EXAMPLE 2

Table 2 Lists the Formula Used in Example 2

TABLE 2
Quantitative Formula

| | | Quantity per | |
|---|---|---|---|
| S.No: | Ingredients | dose [mg] | % w/w |
| 1. | Itraconazole | 100 | 21.74 |
| 2. | Microcrystalline Cellulose | 302 | 65.65 |
| 3. | Croscarmellose sodium | 46.0 | 10.0 |
| 4. | Polyvinyl pyrrolidone K25 | 12.0 | 2.60 |
| 5. | Conc. Hydrochloric acid [37%] | 0.0415 ml | Molar ratio to |
| | | [0.04897 g | drug 1:3.5 moles |
| | | 48.97 mg] | |
| 6. | Ethanol* | — | — |
| 7. | Purified Water* | — | — |
| | Total | 460.0 | |

*Does not appear in final product.

Brief Process of the Invention:
1. Itraconazole is dissolved in the mixture of ethanol, concentrated hydrochloric acid (37%) and purified water.
2. Microcrystalline cellulose, croscarmellose sodium and polyvinyl pyrrolidone K25 are added together and mixed well.
3. The ingredients of step 2 are mixed well and then granulated by the solution of step 1 by top spray technique using a fluid bed granulator.
4. The granules thus obtained can be directly filled into capsules or can be compressed into tablets.

### EXAMPLE 3

Table 3 Lists the Formula Used in Example 3

TABLE 3
Quantitative Formula

| | | Quantity per | |
|---|---|---|---|
| S.No: | Ingredients | dose [mg] | % w/w |
| 1. | Itraconazole | 100.0 | 20.39 |
| 2. | Microcrystalline Cellulose | 135.0 | 27.53 |
| 3. | Croscarmellose | 46.0 | 9.38 |
| | sodium/Crospovidone | | |
| 4. | Hydroxypropyl-β- | 167.0 | 34.05 |
| | cyclodextrin | | (Molar ratio to |
| | | | drug 1:0.9 |
| | | | moles)# |
| 5. | Polyvinyl pyrrolidone K25 | 12.0 | 2.45 |
| 6. | Crospovidone | 28.1 | 5.73 |
| 7. | Conc. Hydrochloric acid [37%] | 0.0415 ml | Molar ratio to |
| | | [48.97 mg] | drug 1:3.5 moles |
| 8. | Ethanol* | — | — |
| 9. | Purified Water* | — | — |
| | Total | 490.4 | |

#Molecular Weight of HP3-β-CD used is 1309
*Does not appear in final product.

Brief Process of the Invention:
1. Itraconazole is dissolved in the mixture of ethanol, concentrated hydrochloric acid (37%) and purified water.
2. Hydroxypropyl-β-cyclodextrin is dissolved in sufficient volume of purified water.
3. The solution of step 1 and step 2 are mixed together and stirred well.
4. Microcrystalline cellulose, croscarmellose sodium/crospovidone and polyvinyl pyrrolidone K25 are added together and mixed well.
5. The ingredients of step 4 are mixed well and then granulated by the solution of step 3 by top spray technique using a fluid bed granulator.
6. Crospovidone and magnesium stearate are added together to the granules and then roll compacted.
7. The roll compacted mass is then sized/milled.
8. Crospovidone is then added to the granules from step 7.
9. The granules are then filled into capsules.
   In Vitro Dissolution Profile Comparison with Sporanox®

The comparative in-vitro dissolution Profiles of the products of Example 1, Example 2, Example 3, and Sporanox®, a marketed form of itraconazole available from Janssen Pharmaceutica Products of Titusville, N.J., are given in Table 4.

Apparatus: USP Type 2

RPM: 100

Medium: 900 ml of Simulated Gastric Fluid (SGF) without enzymes at 37° C.

TABLE 4
Comparative Dissolution Profile

| | % Itraconazole dissolved | | | |
|---|---|---|---|---|
| Time | Sporanox | | | |
| (min) | (B.No. 2JG256) | Example 1 | Example 2 | Example 3 |
| 15 | 27 | 4 | 42 | 55 |
| 30 | 48 | 28 | 62 | 83 |
| 45 | 66 | 49 | 73 | 89 |
| 60 | 79 | 61 | 77 | 93 |

1. A pharmaceutical composition comprising a plurality of non-spherical granules, wherein said granules do not contain a coated core region and further comprise:
a. an antifungal active pharmaceutical ingredient;
b. a bulking agent;
c. a disintegrant;
d. a binding agent;
e. an acid; and,
wherein said antifungal active pharmaceutical ingredient is distributed uniformly throughout the non-spherical granule.

2. The composition of claim 1, wherein said composition is a pharmaceutically acceptable dosage form selected from the group consisting of tablet, capsule and caplet.

3. The composition of claim 1, wherein said antifungal active pharmaceutical ingredient is selected from the group consisting of itraconazole, saperconazole, ketoconazole, voriconazole and fluconazole.

4. The composition of claim 1, wherein said bulking agent is selected from the group consisting of mannitol and microcrystalline cellulose.

5. The composition of claim 1, wherein said disintegrant is selected from the group consisting of croscarmellose sodium, crospovidone, sodium starch glycolate.

6. The composition of claim 1, wherein said disintegrant comprises a mixture of croscarmellose sodium and crospovidone.

7. The composition of claim 1, wherein said binding agent is selected from the group consisting of polyvinyl pyrrolidone and polyvinyl pyrrolidone K25.

8. The composition of claim 1, wherein said acid is hydrochloric acid.

9. The composition of claim 1, wherein said non-spherical granules further comprise a cyclodextrin.

10. The composition of claim 9, wherein said cyclodextrin is hydroxypropyl-β-cyclodextrin.

11. The composition of claim 1, wherein said non-spherical granules further comprise a second disintegrant.

12. The composition of claim 11, wherein said second disintegrant is selected from the group consisting of crospovidone, croscarmellose sodium and sodium starch glycolate.

13. The composition of claim 11, wherein said non-spherical granules further comprise a third disintegrant.

14. The composition of claim 11, wherein said third disintegrant is selected from the group consisting of crospovidone, croscarmellose sodium and sodium starch glycolate.

15. A method for the treatment of fungal infections which comprises administering to a patient in need thereof an effective amount of a dosage form according to claim 1.

16. A pharmaceutical composition comprising a plurality of non-spherical granules, wherein said granules do not contain a coated core region and further comprise:
(a) itraconazole;
(b) a binding agent selected from the group consisting of mannitol and microcrystalline cellulose;
(c) croscarmellose sodium;
(d) polyvinyl pyrrolidone;
(e) and hydrochloric acid; and,
wherein said antifungal active pharmaceutical ingredient is distributed uniformly throughout the non-spherical granule.

17. A method for the treatment of fungal infections which comprises administering to a patient in need thereof an effective amount of a dosage form according to claim 16.

18. A pharmaceutical composition comprising a plurality of non-spherical granules, wherein said granules do not contain a coated core region and further comprise:
(a) itraconazole;
(b) a bulking agent selected from the group consisting of mannitol and microcrystalline cellulose;
(c) a croscarmellose sodium and crospovidone mixture;
(d) crospovidone;
(e) polyvinyl pyrrolidone;
(f) a cyclodextrin; and
(g) hydrochloric acid; and,
wherein said antifungal active pharmaceutical ingredient is distributed uniformly throughout the non-spherical granule.

19. The composition of claim 18, wherein said cyclodextrin is hydroxypropyl-β-cyclodextrin.

20. A method for the treatment of fungal infections which comprises administering to a patient in need thereof an effective amount of a pharmaceutical dosage form according to claim 18.

21. A method for preparing a pharmaceutical dosage form including a plurality of non-spherical granules, wherein said granules do not contain a coated core region, comprising the steps of:
(a) dissolving an antifungal active pharmaceutical ingredient in an alcohol, an acid, and water;
(b) mixing a bulking agent, a disintegrant, and a binding agent to form a base mixture;
(c) granulating the mixture of step (b) with the mixture of step (a); and
(d) forming a pharmaceutical dosage form from the non-spherical granules from step (c); and,
wherein said antifungal active pharmaceutical ingredient is distributed uniformly throughout the non-spherical granule.

22. The method of claim 21, wherein said pharmaceutical dosage form is selected from the group consisting of tablet, capsule and caplet.

23. The method of claim 21, wherein said antifungal active pharmaceutical ingredient is selected from the group consisting of itraconazole, saperconazole, ketoconazole, voriconazole and fluconazole.

24. The method of claim 21, wherein said alcohol is ethanol.

25. The method of claim 21, wherein said acid is hydrochloric acid.

26. The method of claim 21, wherein said bulking agent is selected from the group consisting of mannitol and microcrystalline cellulose.

27. The method of claim 21, wherein said disintegrant is selected from the group consisting of crospovidone, croscarmellose sodium and sodium starch glycolate.

28. The method of claim 21, wherein said binding agent is selected from the group consisting of polyvinyl pyrrolidone and polyvinyl pyrrolidone K25.

29. A method of treatment of fungal infections which comprises administering to a patient in need thereof an effective amount of a dosage form according to claim 21.

30. A method for preparing a pharmaceutical dosage form including a plurality of non-spherical granules, wherein said granules do not contain a coated core region, comprising the steps of:
(a) dissolving an antifungal active pharmaceutical ingredient in an alcohol, an acid, and water;
(b) adding a cyclodextrin dissolved in water to the solution of step (a);
(c) mixing a bulking agent, a first disintegrant, and a binding agent;
(d) granulating the mixture of step (c) with the solution of step
(e) adding a mixture of a second disintegrant and a lubricant to the mixture of step
(f) compacting the mixture of step (e) into a compacted mass;
(g) milling and sizing said compacted mass into non-spherical granules;
(h) adding a third disintegrant to said non-spherical granules; and
(i) forming a pharmaceutical dosage from said non-spherical granules and,
wherein said antifungal active pharmaceutical ingredients is distributed uniformly throughout the non-spherical granule.

31. The method of claim 31, wherein said pharmaceutical dosage form is selected from the group consisting of tablet, capsule and caplet.

32. The method of claim 31, wherein said antifungal active pharmaceutical ingredient is selected from the group consisting of itraconazole, saperconazole, ketoconazole, voriconazole and fluconazole.

33. The method of claim 31, wherein said alcohol is ethanol.

34. The method of claim 31, wherein said acid is hydrochloric acid.

35. The method of claim 31, wherein said cyclodextrin is hydroxypropyl-β-cyclodextrin.

36. The method of claim 31, wherein said bulking agent is microcrystalline cellulose.

37. The method of claim 31, wherein said first disintegrant is selected from the group consisting of crospovidone, croscarmellose sodium and sodium starch glycolate.

38. The method of claim 31, wherein said disintegrant comprises a mixture of croscarmellose sodium and crospovidone.

39. The method of claim 31, wherein said binding agent is selected from the group consisting of polyvinyl pyrrolidone and polyvinyl pyrrolidone K25.

40. The method of claim 31, wherein said second disintegrant is selected from the group consisting of crospovidone, croscarmellose sodium and sodium starch glycolate.

41. The method of claim 31, wherein said lubricant is magnesium stearate.

42. The method of claim 31, wherein said third disintegrant is selected from the group consisting of crospovidone, croscarmellose sodium and sodium starch glycolate.

43. A method of treatment of fungal infections which comprises administering to a patient in need thereof a pharmaceutical dosage form according to claim 31.

44. A method for preparing a pharmaceutical dosage form including a plurality of non-spherical granules, wherein said granules do not contain a coated core region, comprising the steps of:
(a) dissolving itraconazole in ethanol, hydrochloric acid, and water;
(b) mixing a bulking agent selected from the group consisting of mannitol and microcrystalline cellulose, croscarmellose cellulose, and polyvinyl pyrrolidone;
(c) granulating the base mixture of step (b) with the solution of step (a); and
(d) forming a pharmaceutical dosage from the non-spherical granules of step (c) and,
wherein said antifungal active pharmaceutical ingredient is distributed uniformly throughout the non-spherical granule.

45. A method of treatment of fungal infections which comprises administering to a patient in need thereof a dosage form according to claim 44.

46. A method for preparing a pharmaceutical dosage form including a plurality of non-spherical granules, wherein said granules do not contain a coated core region, comprising the steps of:
(a) dissolving itraconazole in ethanol, hydrochloric acid, and water;
(b) adding a cyclodextrin dissolved in water to the solution of step (a);
(c) mixing microcrystalline cellulose, a croscarmellose sodium and crospovidone mixture, and polyvinyl pyrrolidone;
(d) granulating the mixture of step (c) with the solution of step (b);
(e) adding a mixture of a crospovidone and magnesium stearate to the granules of step (c);
(f) compacting said granules into a compacted mass;
(g) milling and sizing said compacted mass into non-spherical granules;
(h) adding crospovidone to said non-spherical granules; and
(i) forming a pharmaceutical dosage from said non-spherical granules; and,
wherein said antifingal active pharmaceutical ingredient is distributed uniformly throughout the non-spherical granule.

47. The method of claim 46, wherein said cyclodextrin is hydroxypropyl-β-cyclodextrin.

48. A method of treatment of fungal infections which comprises administering to a patient in need thereof a dosage form according to claim 46.
